(19) United States
(12) Patent Application Publication
(10) Pub. No.: US 2022/0230712 A1
(43) Pub. Date: Jul. 21, 2022

(54) SYSTEMS AND METHODS FOR TEMPLATE-FREE REACTION PREDICTIONS

(71) Applicant: Kebotix, Inc., Cambridge, MA (US)

(72) Inventors: Dennis Sheberla, Bedford, MA (US); Christoph Kreisbeck, Cambridge, MA (US); Kevin Ryan, Watertown, MA (US); Chandramouli Nyshadham, Watertown, MA (US); Hengyu Xu, Lexington, MA (US)

(73) Assignee: Kebotix, Inc., Cambridge, MA (US)

(21) Appl. No.: 17/579,844

(22) Filed: Jan. 20, 2022

Related U.S. Application Data
(60) Provisional application No. 63/140,090, filed on Jan. 21, 2021.

(51) Int. Cl. G16C 20/10; G16C 20/70; G16C 20/80

(52) U.S. Cl. CPC G16C 20/10; G16C 20/80; G16C 20/70

(57) ABSTRACT

The techniques described herein relate to methods and apparatus for determining a set of reactions to produce a target product. The method includes receiving the target product, executing a graph traversal thread, requesting, via the graph traversal thread, a first set of reactant predictions for the target product, executing a molecule expansion thread, determining, via the molecule expansion thread and a reactant prediction model, the first set of reactant predictions, and storing the first set of reactant predictions as at least part of the set of reactions.

### RELATED APPLICATIONS

This Application claims the benefit under 35 U.S.C. § 119(e) of U.S. Provisional Application Ser. No. 63/140,090, filed on Jan. 21, 2021, entitled “SYSTEMS AND METHODS FOR TEMPLATE-FREE REACTION PREDICTIONS,” which is incorporated herein by reference in its entirety.

### FIELD

This application relates generally to template-free techniques for predicting reactions.

### BACKGROUND

The exploration of the chemical space is central to many areas of research, such as drug discovery, material synthesis, and biomolecular chemistry. Chemical exploration can be a challenging problem because the space of possible transformations is vast and it requires experienced chemists. The discovery of novel chemical reactions and synthesis pathways is a perennial goal for synthetic chemists, but it requires years of knowledge and experience. It is therefore desirable to provide new technologies that can support the creativity of chemists in synthesizing novel molecules with enhanced properties, including providing chemistry prediction tools to assist chemists in various synthesis tasks such as reaction prediction, retrosynthesis, agent suggestion, and/or the like.

### SUMMARY

According to one aspect, a computerized method is provided for determining a set of reactions (e.g., a chemical reaction network or graph) to produce a target product. The method includes receiving the target product, executing a graph traversal thread, requesting, via the graph traversal thread, a first set of reactant predictions for the target product, executing a molecule expansion thread, determining, via the molecule expansion thread and a reactant prediction model (e.g., a single-step retrosynthesis model), the first set of reactant predictions, and storing the first set of reactant predictions as at least part of the set of reactions.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should be further appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

Various aspects and embodiments will be described herein with reference to the following figures. It should be appreciated that the figures are not necessarily drawn to scale. Items appearing in multiple figures are indicated by the same or a similar reference number in all the figures in which they appear.

FIG. 1 is a diagram of an exemplary system for providing template-free reaction predictions, according to some embodiments.

FIG. 2 is a diagram of an exemplary reaction prediction flow, according to some embodiments.

FIG. 3A is a diagram showing generation of a reaction network graph in the chemical space using retrosynthesis, according to some embodiments.

FIG. 3B is a diagram of another example of generating a reaction network graph in the chemical space, according to some embodiments.

FIG. 4 is a diagram of the aspects of an exemplary model prediction process, according to some embodiments.

FIG. 5 is a diagram showing an exemplary computerized method for determining a set of reactions to produce a target product, according to some embodiments.

FIG. 6 is a diagram of exemplary strings that can be used for reaction predictions, according to some embodiments.

FIG. 7 is a diagram of an exemplary computerized process for single-step retrosynthesis prediction using forward and reverse models, according to some embodiments.

FIG. 8 shows a block diagram of an exemplary computer system that may be used to implement embodiments of the technology described herein.

### DETAILED DESCRIPTION

Retrosynthesis aims to identify a series of chemical transformations for synthesizing a target molecule. In a single-step retrosynthesis formulation, the task is to identify a set of reactant molecules for a given a target. Conventional retrosynthesis prediction techniques often require looking up transformations in databases of known reactions. The vast space of possible chemical transformations makes retrosynthesis a challenging problem and typically requires the skill of experienced chemists. Synthesis planning requires chemists to visualize the end-product and work backward toward increasingly simpler compounds. Synthesizing novel pathways is a challenging task as it depends on the optimization of many factors, such as the number of intermediate steps, available starting materials, cost, yield, toxicity, and/or other factors. Further, for many target compounds, it is possible to establish alternative synthesis routes, and the goal is to discover reactions that will affect only one part of the molecule, leaving other parts unchanged.

Synthesis planning may also require the ability to extrapolate beyond established knowledge, which is typically not possible using conventional techniques that rely on databases of known reactions. The inventors have appreciated that data-driven AI models can be used to attempt to add such reasoning with the goal of discovering and/or rediscovering new transformations. AI models can include template-based models (e.g., deep learning approaches with symbolic AI, graph convolutional networks, etc.) and template-free models (e.g., molecular transformer models). Template-based models can be built by learning the chemical transformations (e.g., templates) from a database of reactions, and can be used to perform various synthesis tasks such as forward reaction prediction or retrosynthesis. Template-free models can be based on machine-translation models (e.g., those used for natural language processing) and can therefore be trained using text-based reactions (e.g., input in Simplified Molecular-Input Line-Entry System (SMILES) notation).

Molecules and chemical reactions can be represented as a chemical reaction network or graph, in which molecules correspond to nodes and reactions to directed connections between these nodes. The reactions may include any type of chemical reaction, e.g., that involve changes in the positions in electrons and/or the formation or breaking of chemical bonds between atoms, including but not limited to changes in covalent bonds, ionic bonds, coordinate bonds, van der Waals interactions, hydrophobic interactions, electrostatic interactions, atomic complexes, geometrical configurations (e.g., molecules contained in molecular cages), and the like. The inventors have discovered and appreciated that template-free models can be used to build such networks. In particular, template-free models can provide desired flexibility because such models need not be restricted by the chemistry (e.g., transformation rules) within the dataset. Additionally, or alternatively, template-free models can extrapolate in the chemical space by learning the correlation between chemical motifs in the reactants and products specified by text-based reactions. However, building chemical reaction networks using template-free models can suffer from various deficiencies. For example, techniques may require identifying molecules for expansion and also expanding those molecules to build out the chemical reaction network. However, if such processing tasks are not able to be decoupled, it can add significant overhead and inefficiencies in building chemical reaction networks. The inventors have therefore developed techniques for determining a set of reactions (e.g., a chemical reaction network or graph) to produce a target product that leverage various threads to distribute the processing required to determine the set of reactions. In some embodiments, a graph traversal thread is used to iteratively identify molecules for expansion to develop a chemical network that can be used to ultimately make the target product. One or more molecule expansion threads can be used to run prediction model(s) (e.g., single-step retrosynthesis models) to determine reactant predictions for molecules identified for expansion by the graph traversal thread. Multiple molecule expansion threads can be run depending on the number of requests from the graph traversal thread. The iterative execution of the graph traversal thread and molecule expansion threads can result in efficient and robust techniques for ultimately determining a set of reactions to build a target product.

The inventors have further discovered and appreciated problems with conventional techniques used to train such models. In particular, large datasets are often used to train the models. For some training sets, such as image-based data sets, the data can be augmented for training. For example, training approaches for image recognition models can include performing augmentations such as random rotations, skews, brightness, and contrast adjustments (e.g., because such augmentations should not affect the presence of the object that an image contains that is to be recognized). However, the inventors have appreciated that there is a need to augment other types of training data, such as non-image-based training sets (e.g., which can be used for text-based models). In particular, the inventors have appreciated that there is no analogy to such image-based augmentations for text-based models, and therefore existing text-based platforms do not provide augmentation tools for text-based inputs (and may not even allow for addition of augmentation techniques).

The inventors have further appreciated that data augmentation can impose large storage requirements. For example, conventional augmentation approaches often require generating a number of different copies of the dataset (e.g., so that the model has sufficient data to process over the course of training). However, since the copies need to be stored during training, and the training process may run for days or weeks, such conventional approaches can have a big impact on storage. For example, if it takes an hour to loop through all training examples and the model converges over the course of three days, then conventional approaches would need to create seventy two (24*3) copies of the training set in order to have the equivalent example diversity from data augmentation. To further illustrate this point, if the training time is increased by a factor of five, then the storage requirements would likewise be five times larger (e.g., three hundred and sixty copies (24*3*5) of the dataset).

The inventors have therefore developed an input augmentation pipeline that provides for iterative augmentation techniques. The techniques provide for augmenting text-based training data sets, including to vary the input examples to improve the robustness of the model. The techniques further provide for augmenting subsets of the training data and using the subsets to iteratively train the model while further subsets are augmented. The techniques can drastically reduce the storage requirements since significantly less data needs to be stored using the iterative approach described herein compared to conventional approaches. Such techniques can be used to train both forward prediction models and reverse prediction models, which can be run together for single-step retrosynthesis prediction in order to validate results predicted by each model.

Although particular exemplary embodiments of the template-free models will be described further herein, other alternate embodiments of all components related to the models (including training the models and/or deploying the models) are interchangeable to suit different applications. Turning to the figures, specific non-limiting embodiments of template-free models and corresponding methods are described in further detail. It should be understood that the various systems, components, features, and methods described relative to these embodiments may be used either individually and/or in any desired combination as the disclosure is not limited to only the specific embodiments described herein.

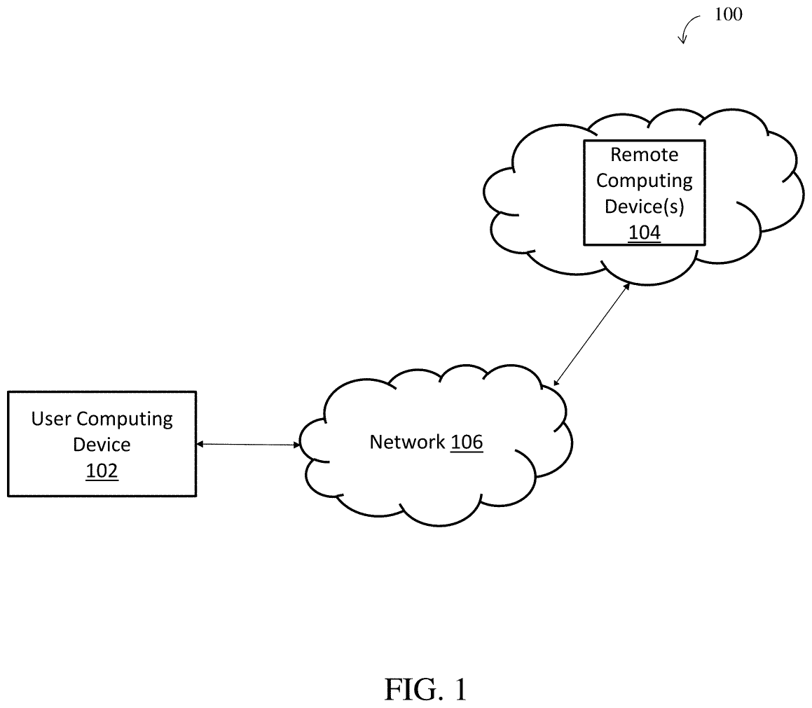

In some embodiments, the techniques can provide a tool, such as a portal or web interface, for performing chemical reaction predictions. In some embodiments, the tool can be provided by one or more computing devices that serve one or more web pages to users. The web pages can be used to collect data required to perform the computational aspects of the predictions. FIG. 1 is a diagram of an exemplary system 100 for providing template-free reaction predictions, according to some embodiments. The system 100 includes a user computer device 102 that is in communication with one or more remote computing devices 104 through network 106. The user computing device 102 can be any computing device, such as a smart phone, laptop, desktop, and/or the like. The one or more remote computing devices 104 can be any suitable computing device used to provide the techniques described herein, and can include a desktop or laptop computer, web server(s), data server(s), back-end server(s), cloud computing resources, and/or the like. As described herein, the remote computing devices 104 can provide an online tool that allows users to perform chemical predictions, high throughput screening, and/or synthesizability prediction for molecules, according to the techniques described herein.

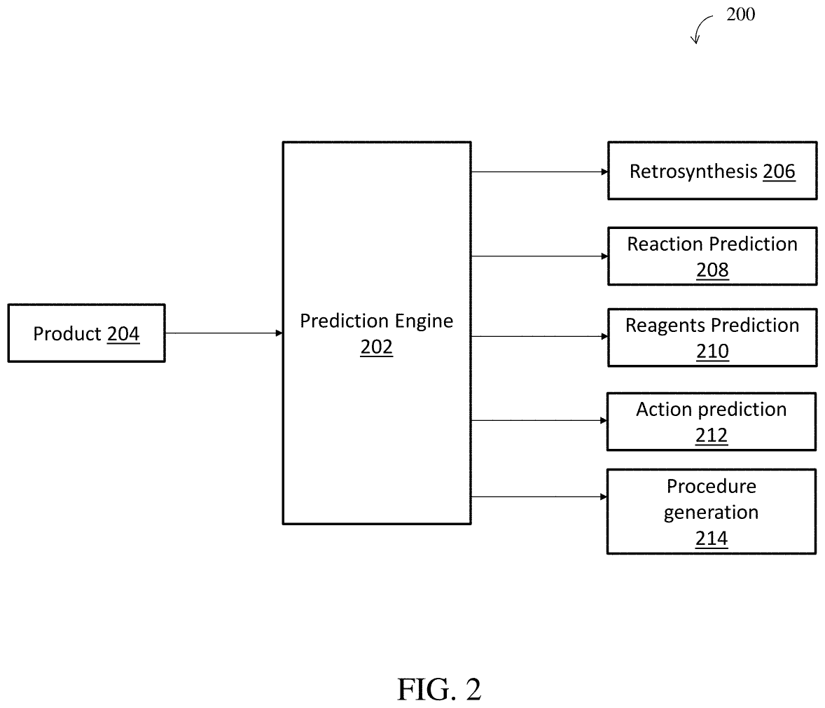

FIG. 2 is a diagram of an exemplary reaction prediction flow 200, according to some embodiments. The prediction engine 202 receives an input/desired product 204 and can perform one or more of a retrosynthesis analysis 206, reaction prediction 208, and/or reagents prediction 210. As described herein, the prediction engine 202 can build a chemical reaction network based on the product 204 (e.g., a target molecule) to model the behavior of real-world chemical systems. The prediction engine 202 can analyze the reaction graph to assist chemists in various tasks such as retrosynthesis 206. For example, the prediction engine can analyze the graph using various algorithms as described herein for tasks such as forward reaction prediction. The prediction engine 202 can also provide for reaction prediction 208 and/or reagents prediction 210, such as by leveraging a transformer model as described further below.

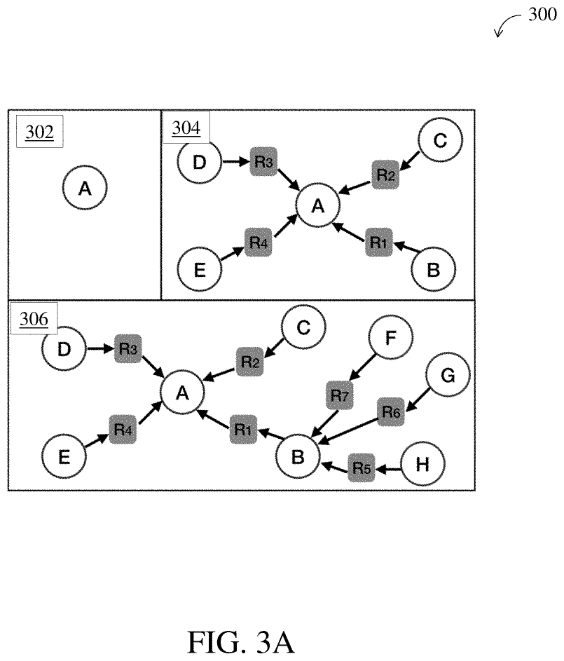

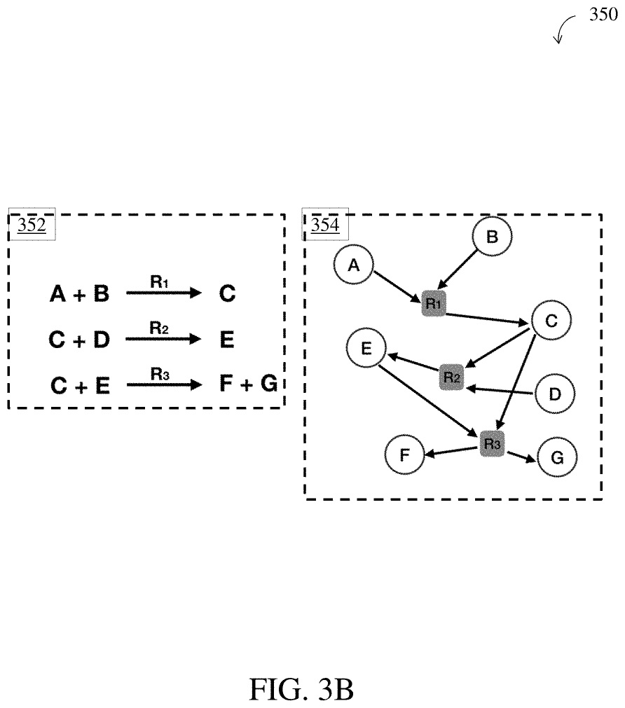

In some embodiments, the prediction engine 202 can send a list of available options to users (e.g., via a user interface). Users can configure the options for queries to the prediction engine 202. For example, the system may use the options to dynamically generate parts of the graphical user interface. As another example, the options can allow the prediction engine 202 to receive a set of configured options that allow users to modify parameters related to their queries and/or predictions. Examples of configurable options include prediction runtime, additional feedstock, configurations to control model predictions (e.g., desired number of routes, maximum reactions in a route, molecule/reaction blacklists, etc.), and/or the like. In some embodiments, the prediction engine 202 can generate the reaction network graphs for each prediction. The molecules can be pre-populated and/or populated per a chemist's requirements. In some embodiments, given a target molecule, reaction, or reagents, the prediction engine can generate the reaction network through a series of single-step retrosynthesis steps starting from the input molecule. FIG. 3A is a diagram 300 showing a simplified example of generating a reaction network graph in the chemical space using the retrosynthesis, according to some embodiments. Given a target molecule A 302, the prediction engine generates the reaction network through a series of single-step retrosynthesis, as shown in 304 and 306. In some embodiments, the input target molecule and feedstock molecules can be specified in text string-based notations, such as SMILES notation, or others such as those described herein. As shown in 304, a first retrosynthesis step generates molecules ‘B,’ ‘C,’ ‘D,’ and ‘E’ in the graph, which are associated with reagents R₁, R₂, R₃ and R₄, respectively. The graph traversal algorithm then chooses the next target (molecule B, in this example) and performs another single step retrosynthesis, thus generating the graph reaction network until the desired synthesis path is found. The graph 306 therefore further includes molecules ‘F,’ ‘G,’ and ‘H’ in the graph, which are associated with reagents R₇, R₆, and R₅, respectively. The arrowheads in 304 and 306 indicate the direction of the reaction. It should be appreciated that the graph shown in FIG. 3A is for exemplary purposes, and that in practice the graphs can be significantly larger. For example, the techniques are capable of producing large reaction network graphs generating reactions at the rate of >5000 reactions/minute on average (e.g., around 5000 reactions/minute per GPU, which can therefore be scaled according to the number of GPUs).

FIG. 3B is a diagram 350 of another example of generating a reaction network graph in the chemical space, according to some embodiments. Section 352 shows three example reactions where A, B, C, D, E, F, G are compounds, and R₁-R₃ are reagents. Section 354 shows a graph network of the chemical reactions shown in section 352, where the molecules A, B, C, D, E, F, G correspond to nodes, and reactions correspond to directed connections between these nodes like with FIG. 3A.

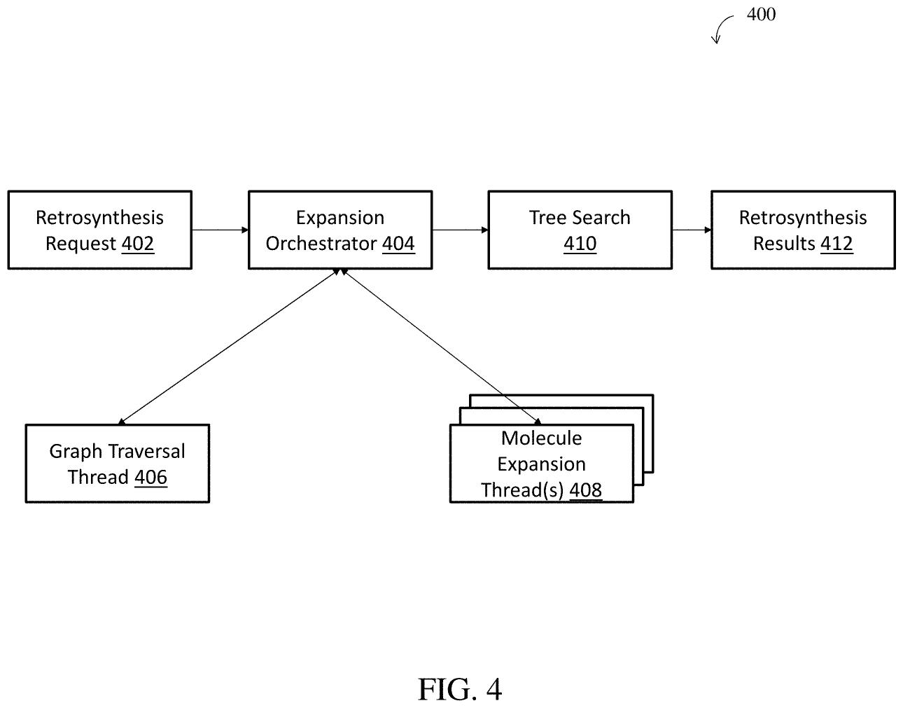

The techniques described herein can be used to perform retrosynthesis for target molecules to identify a set of reactions that can be used to build the target molecules. FIG. 4 is a diagram of the aspects of an exemplary model prediction process 400, according to some embodiments. As described herein, the prediction process can be performed using, for example, a template-free model. As shown, the model prediction process includes a retrosynthesis request 402, an expansion orchestrator 404 (which coordinates the graph traversal thread 406 and the molecule expansion thread(s) 408), a tree search 410, and retrosynthesis results 412.

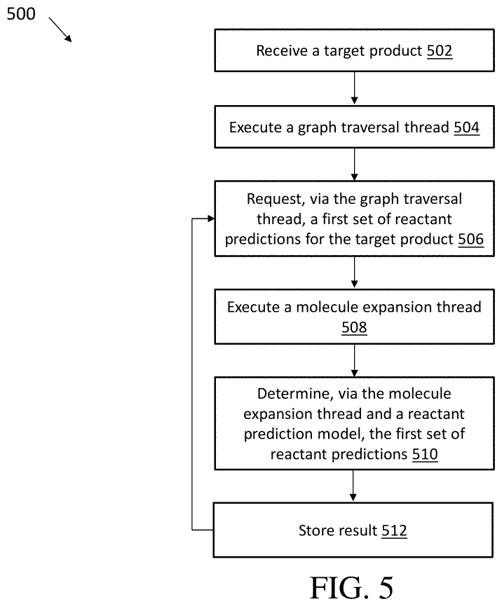

FIG. 4 will be described in conjunction with FIG. 5, which is a diagram showing an exemplary computerized method 500 for determining a set of reactions (e.g., a chemical reaction network or graph) to produce a target product, according to some embodiments. At step 502, the prediction engine receives the target product for the retrosynthesis request 402. At step 504, the expansion orchestrator 404 executes the graph traversal thread 406. At step 506, the prediction engine requests, via the graph traversal thread 406, a first set of reactant predictions for the target product. In response, at step 508 the expansion orchestrator 404 executes a molecule expansion thread 408. At step 510, the prediction engine determines, via the molecule expansion thread 408 and a reactant prediction model (e.g., a single-step retrosynthesis model), the first set of reactant predictions. At step 512, the prediction engine stores the first set of reactant predictions as at least part of the set of reactions.

The method 500 proceeds back to step 506 and performs further predictions on the results determined at step 510 to build the full set of results (e.g., to build a full chemical reaction network). For example, referring to FIG. 3A, the first execution of steps 506 through 512 on molecule A 302 can generate the portion of the graph shown in 304, with molecules ‘B,’ ‘C,’ ‘D,’ and ‘E’ in the chemical network (and reagents R₁, R₂, R₃ and R₄, respectively). A second iteration of steps 506 through 512 can be performed on the next target (molecule B, in this example) to perform another single step retrosynthesis, thus generating the graph 306, which further includes molecules ‘F,’ ‘G,’ and ‘H’ in the graph (and reagents R₇, R₆, and R₅, respectively) that stem from molecule B.

Once built, the prediction engine performs a tree search (e.g., 410 in FIG. 4), and ultimately generates the retrosynthesis results 412 that are provided to the user in response to the retrosynthesis request 402. The tree search 410 can be used to identify a plurality of different ways that the target molecule can be built based on the chemical reaction network or graph. For example, referring further to FIG. 3A, any of ‘B,’ ‘C,’ ‘D,’ and ‘E’ in the chemical network (and reagents R₁, R₂, R₃ and R₄, respectively) can be used to build the target molecule A 302. If molecule ‘B’ is chosen, then there are three further options available to build ‘B:’ one option is to use molecule ‘F’ and reagent R₇, a second option is to use molecule ‘G’ and reagent R₆, and a third option is to use molecule ‘H’ and reagent R₈. As a result, the retrosynthesis results 412 can include a listing of different techniques that can be used to build the target product.

The inventors have appreciated that the set of results (e.g., a retrosynthetic graph) may contain a number of routes that differ in chemically insignificant ways. An example of this is two routes that only differ by using different solvents in one of the reactions. In some embodiments, the results may be especially prone to such a problem, since the techniques can include directly predicting solvents and other related details. In some embodiments, such insignificantly-differing routes can be addressed using modified searching strategies. For example, the techniques can include repeatedly calling a tree search to find the “best” (e.g., according to an arbitrary/interchangeable criteria that can be specified or configured) route in the retrosynthetic graph. After each tree search, a blacklist for reactant-product pairs can be created from some and/or all reactions in the returned route. Each successive tree search can be prohibited from using some and/or all of the reactions that contain a reaction-product pair found in the blacklist. This search process can be repeated, for example, until a requested number of routes are found, the process times out, and/or all possible trees in the retrosynthetic graph are exhausted.

It should be appreciated that while a tree search is discussed herein as an exemplary technique for identifying the retrosynthesis results, other types of searches can be used with the techniques described herein. Other exemplary search strategies include, for example, depth-first search, breadth-first search, iterative deepening depth-first search, and/or the like. In some embodiments, the results (e.g., the chemical reaction network) can be preprocessed prior to the search. Pruning can be performed prior to tree search, during the retrosynthesis expansion loop (e.g., by the expansion orchestrator 404), and/or the like. For example, a pruning process can be performed on the results prior to the search to prune reactions based on a determination of whether they can be part of the best route. Reactions may be pruned, for example, if reactions require stock outside of a specified list, if reactions can't produce a complete route (e.g., with all starting materials in feedstock), reactions include blacklisted molecules, blacklisted reactions, reactions with undesirable properties (e.g., solubility of intermediates, reaction rate, reaction enthalpy, thermodynamics, etc.), and/or the like.

The graph traversal thread 406 can be used by the expansion orchestrator 404 to repeatedly build out routes (e.g., branches) of the chemical reaction network by analyzing predicted reactions from a particular step to identify molecules to further expand in subsequent steps. The expansion orchestrator 404 can frequently communicate with the expansion orchestrator 404, such as once every few milliseconds. The graph traversal thread 406 can send molecule expansion requests to the expansion orchestrator 404, and can retrieve retrosynthesis graph updates made by the expansion orchestrator 404.

In some embodiments, the expansion orchestrator 404 can be executed as a separate thread or process from the graph traversal thread 406 and the molecule expansion thread(s) 408, can coordinate the graph traversal thread 406 and the molecule expansion thread(s) 408. Generally, the expansion orchestrator 404 can (repeatedly) execute the graph traversal thread 406, and can provide a list of reactions (e.g., as a string) and confidences (e.g., as numbers, such as floats), as necessary, to the graph traversal thread 406. The expansion orchestrator 404 can receive molecule expansion requests from the graph traversal thread 406 for reactant predictions of new molecules (e.g., the target product and/or other molecules determined through the prediction process). The expansion orchestrator 404 can coordinate execution of the molecule expansion thread(s) 408 accordingly to determine reactant predictions requested by the graph traversal thread 406. As an illustrative example, in some embodiments the expansion orchestrator 404 can leverage queues, such as Python queues, to coordinate with the graph traversal worker 406. As another example, the expansion orchestrator 404 can leverage Dask futures to provide for real-time execution of the molecule expansion threads 408. However, it should be appreciated that Python and Dask are examples only and are not intended to be limiting.

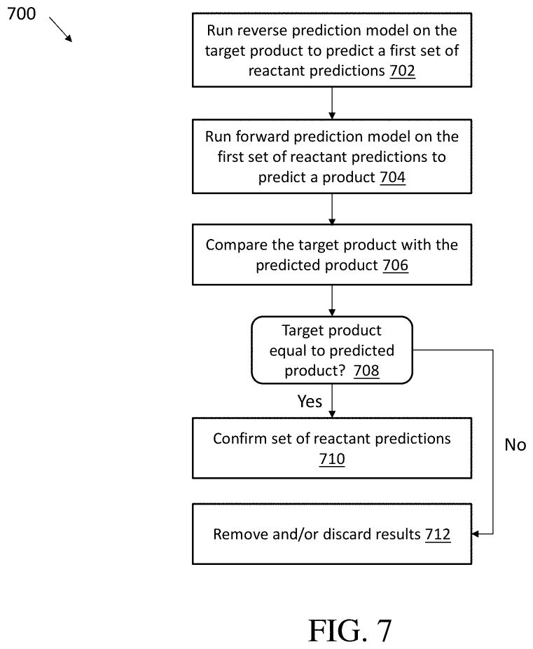

The expansion orchestrator 404 can maintain a necessary number of ongoing expansion requests to molecule expansion thread(s) 408. For each expansion request from the graph traversal thread 406, the expansion orchestrator 404 can execute an associated molecule expansion thread 408 to perform the molecule expansion process to identify new sets of reactant predictions to build out the chemical reaction network. To generate reactant predictions for each molecule expansion request, the molecule expansion thread(s) 408 can each perform single-step retrosynthesis prediction as described in conjunction with FIG. 7. The expansion orchestrator 404 can provide to each molecule expansion thread 408 the molecule for expansion (e.g., as a string), the model path (e.g., as a string), and/or options (e.g., as strings and/or numbers, such as floats or integers) for the expansion process. Each molecule expansion thread 408 can provide a list of reactions (e.g., as a string) and confidences (e.g., as floats) to the expansion orchestrator. The expansion orchestrator 404 can retrieve and accumulate molecule expansion results from the molecule expansion threads 408 as they perform the requested expansions issued from the graph traversal thread 406. The expansion orchestrator 404 can update and maintain a master copy of the retrosynthesis network or graph by adding new expansion results upon receipt from the molecule expansion threads 408. The expansion orchestrator 404 can send retrosynthesis graph updates to the graph traversal thread 406 for consideration for further expansion.

In some embodiments, the expansion process leveraged by the molecule expansion threads 408 can be configured to perform reaction prediction and retrosynthesis using natural language (NL) processing techniques. In some embodiments, the template free model is a machine translation model, or a transformer model. Transformer models can be used for natural language processing tasks, such as translation and autocompletion. An example of a transformer model is described in Segler, M., Preuss, M. & Waller, M. P., “Towards ‘Alphachem’: Chemical synthesis planning with tree search and deep neural network policies,” 5^{th} International Conference on Learning Representations, ICLR 2017—Workshop Track Proceedings (2019), which is hereby incorporated herein by reference in its entirety. Transformer models can be used for reaction prediction and single-step retrosynthesis problems in chemistry. The model can therefore be designed to perform reaction prediction using machine translation techniques between strings of reactants, reagents and products. In some embodiments, the strings can be specified using text-based representations such as SMILES strings, or others such as those described herein.

In some embodiments, the techniques can be configured to use one or a plurality of retrosynthesis models. In some embodiments, the system can execute multiple instances of the same model. In some embodiments, they system can execute multiple different models. The expansion orchestrator 404 can be configured to communicate with the one or a plurality of retrosynthesis models. In some embodiments, if using multiple single-step retrosynthesis models, the expansion orchestrator 404 can be configured to route expansion requests to the multiple models. For example, each expansion request may be routed to a subset and/or all running models. When running multiple of the same models (e.g., alone and/or in combination with other different models), the expansion orchestrator 404 can be configured to route expansion requests to all of the same models. When running different models, expansion requests can be routed based on the different models. For example, expansion requests can be selectively routed to certain model(s), such as by using routing rules and/or routing model(s) that can be configured to send expansion requests to appropriate models based on the expansion requests (e.g., only to those models with applicable characteristics, such as necessary expertise, performance, throughput, etc. characteristics).

In some embodiments, different single-step retrosynthesis models can be generated using the same neural network architecture and/or different neural network architectures. For example, the same neural network architecture and algorithm (e.g., as described in conjunction with FIG. 7) can be used for multiple models, but using different training data to achieve the different models. As another example, the single-step retrosynthesis models may include different model architectures and algorithms. For example, a single-step prediction model could be configured to perform a database lookup to stored reactions (e.g., known reactions). Each single-step retrosynthesis model (e.g., regardless of the model structure, network, and/or algorithm) can be configured to take products as input and return suggested reactions (and associated confidences) as output. As a result, the system can be configured to interact with each model regardless of the model architecture and/or algorithm.

In some embodiments, the molecule expansion threads 408 can be configured to run the multiple models. For example, one or more molecule expansion threads 408 can be run for each of a plurality of models. In some embodiments, the molecule expansion threads 408 can run different models as described herein. The techniques can be configured to scale molecule expansion threads 408 when using multiple models. For example, if two model expansion threads 408 are each configured to run different models, the techniques can include performing load balancing based on requests routed to the different molecule expansion threads 408. For example, if a first model is routed more predictions than a second model, then the system can create more molecule expansion threads 408 for the first model relative to the second model in order to handle the asymmetric demand for predictions and thus achieve load balancing for the models.

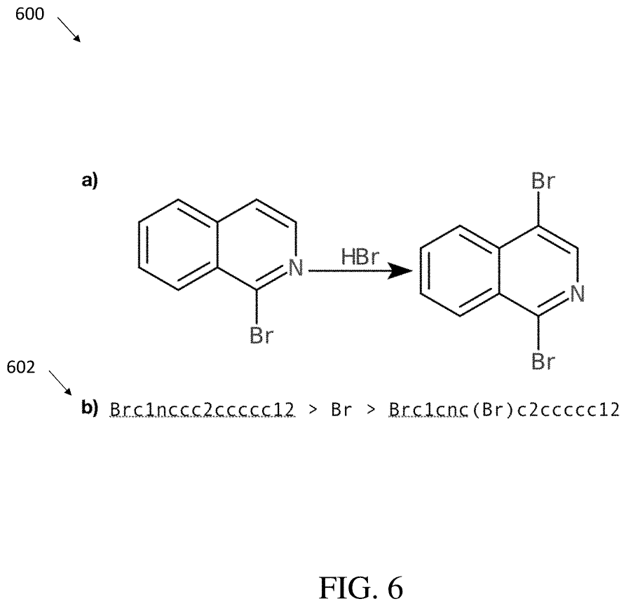

FIG. 6 is a diagram 600 of exemplary strings that can be used for training models for reaction predictions, according to some embodiments. The example in diagram 600 includes a string 602 in SMILES notation of the illustrated reaction. As shown in string 602, reactants, reagents, and products can be delimited using a greater than (>) symbol. As a result, the template-free model need not be restricted to available transformations, and can therefore be capable of encompassing a larger chemical space.

In some embodiments, the trained machine learning model is a trained single-step retrosynthesis model that determines a set of reactant predictions based on the target product. In some embodiments, the model can include multiple models. In some embodiments, the single-step retrosynthesis model includes a trained forward prediction model configured to generate a product prediction based on a set of input reactants, and a trained reverse prediction model configured to generate a set of reactant predictions based on an input product. As a result, the input product can be compared with the predicted product to validate the set of reactant predictions.

Different route discovery strategies can be used for the models, such as using a beam search to discover routes and/or using a sampling strategy to discover routes.

In some embodiments, the reverse prediction model can be configured to leverage a sampling strategy instead of a beam search, since a beam search can (e.g., significantly) limit the diversity of the discovered retrosynthetic routes since many of the predictions produced by beam search are similar to one another from a chemical standpoint. As a result, leveraging a sampling strategy can improve the quality and effectiveness of the overall techniques described herein. For example, sequence models can predict a probability distribution over the possible tokens at the next position and as a result must be evaluated repeatedly, building up a sequence one token at a time (e.g., which can be referred to as decoding). An example of a naïve strategy is greedy decoding, where the most likely token (as evaluated by the model) is selected at each iteration of the decoding process. Beam search can extend this approach by maintaining a set of the k most likely predictions at each iteration (e.g., where k can be referred to as beams). Note that if k=1, the beam search is essentially the same as greedy decoding. In contrast, sampling involves randomly selecting tokens weighted by their respective probability (e.g., sampling from a multinomial distribution). The probabilities of tokens can also be modified with a “temperature” parameter which adjusts the relative likelihood of low and high probability tokens. For example, a temperature of 0 reduces the multinomial distribution to an argmax while an infinite temperature reduces to a uniform distribution. In practice, higher temperatures reduce the overall quality of predictions but increase the diversity. The forward prediction model can use greedy decoding, since the most likely prediction usually has most of the probability density (e.g., since there is usually only 1 possible product in a reaction). The reverse model can use a sampling scheme to generate a variety of possible reactants/agents to make a given product. Regarding the sampling temperatures, temperatures around and/or slightly below 1 (e.g., 0.7, 0.75, 0.8, 0.85) can be used, although the techniques are not so limited (e.g., temperatures up to 1.5, 2, 2.5, 3, etc. can be used as well). Temperatures may be larger or smaller depending on many factors, such as the duration of training, the diversity of the training data, etc.

In some embodiments, a plurality of decoding strategies can be used for the forward and/or reverse prediction models. The decoding strategy can be changed and/or modified at any point (or points) while predicting a sequence using a given model. For example, in some embodiments a first decoding strategy can be used for a first portion of the prediction model, and a second decoding strategy can be used for a second portion of the prediction model (and, optionally, the first and/or a third decoding strategy can be used for a third portion of the prediction model, and so on). As an illustrative example, one decoding strategy can be used to generate one output (e.g., reactants or agents (reagents, solvents and/or catalysts)) and another decoding strategy can be used to generate a second output (e.g., the other of the reactants or agents that is not generated by the first decoding strategy). In particular, sampling can be used to generate reactant molecule(s), and then the sequence can be completed using greedy decoding to generate the (e.g., most likely) remaining set of reactant(s) and reagent(s). However, it should be appreciated that these examples are provided for illustrative purposes and are not intended to be limiting, as other decoding strategies can be used (e.g., beam search) and/or more than two decoding strategies can be used in accordance with the techniques described herein.

In some embodiments, the training process can be tailored based on the search strategy. For example, if the reverse prediction model uses a sampling strategy (e.g., instead of a beam search), then the techniques can include increasing the training time of the reverse prediction model. In particular, the inventors have appreciated that extended training can continue to improve the quality of predictions produced by sampling, even though extended training may not significantly affect the quality of samples produced by other search strategies such as beam search.

FIG. 7 is a diagram of an exemplary computerized process 700 for single-step retrosynthesis prediction using forward and reverse models, according to some embodiments. In some embodiments, the computerized process 700 can be executed by a molecule expansion thread. At step 702, the prediction engine predicts, by running the trained reverse prediction model on the target product, a set of reactant predictions (e.g., a set of reagents, catalysts, and/or solvents). At step 704, the prediction engine predicts, by running the trained forward prediction model on the set of reactant predictions, a product. At step 706, the prediction engine compares the target product with the predicted product. If the comparison shows that the predicted product matches the input product, at step 710 the prediction engine can confirm the set of reactant predictions and store the set of reactant predictions as part of the chemical reaction network. Otherwise, at step 712 the prediction engine can remove and/or discard the results when the predicted product does not match the input product.

In some embodiments, the models described herein can be trained on reactions provided in patents or other suitable documents or data sets, e.g., reactions described in US patents. Any data set may be used, and/or more than one type of data set may be combined (e.g., a proprietary data set with reactions described in US and/or PCT patents and patent applications). In some experiments conducted by the inventors, for example, exemplary models were trained on more than three million reactions described in US patents. The model can be configured to work with any byte sequence that represents the structure of the molecule. The training data set can therefore be specified using any byte matrix or byte sequence, including of arbitrary rank (e.g., one-dimensional sequences (rank-1 matrices) and/or higher dimensional sequences (e.g., two-dimensional adjacency matrices), etc.). Nonlimiting examples include general molecular line notation (e.g., SMILES, SMILES arbitrary target specification (SMARTS), Self-Referencing Embedded Strings (SELFIES), SMIRKS, SYBYL Line Notation or SLN, InChI, InChIKey, etc.), connectivity (e.g., matrix, list of atoms, and list on bonds), 3D coordinates of atoms (e.g., pdb, mol, xyz, etc.), molecular subgroups or convolutional formats (e.g., fingerprint, neural fingerprint, morgan fingerprint, RDKit fingerprinting, etc.), Chemical Markup Language (e.g., ChemML or CML), JCAMP, XYZ File Format, and/or the like. In some embodiments, the techniques can convert the input formats prior to training. For example, a table search can be used to convert convolutional formats, such as to convert InChIKey to InChI or SMILES. As a result, the predictions can be based on learning, through training, the correlations between the presence and absence of chemical motifs in the reactants, reagents, and products present in the available data set.

In some embodiments, the techniques can include providing one or more modifications to the notation(s). The modifications can be made, for example, to account for possible ambiguities in the notation, such as when multi-species compounds are written together. Using SMILES as an illustrative example not intended to be limiting, the SMILES encoding can be modified to group species in certain compounds (e.g., ionic compounds). Reaction SMILES uses a “.” symbol as a delimiter separating the SMILES from different species/molecules. Ionic compounds are often represented as multiple charged species. For example, sodium chloride is written as “[Na+].[Cl—]”. This can cause ambiguity when multiple multi-species compounds are written together. An example of such an ambiguity is a reaction with sodium chloride and potassium perchlorate. Depending on how the canonical order is specified, the SMILES could be “[O—][Cl+3]([O—])([O—])[O—].[Na+].[Cl—].[K+]”. However, with such an order, it is not possible to tell if the species added were sodium chloride and potassium perchlorate, or potassium chloride and sodium perchlorate.

Accordingly, reaction SMILES can be modified to use different characters to delimit the species in multi-species compounds and molecules. Any character not currently used in the SMILES standard, for example, could be used (e.g., a space “ ”). As a result, a model trained on this modified representation can allow the system to determine the proper subgrouping of species in reaction SMILES. Further, the techniques can be configured to revert back to the original form of the notation. Continuing with the previous example, the conventional reaction SMILES convention can be reverted back to by replacing occurrences of the molecule/species delimiters (e.g., spaces “ ”, in this example) with the standard character molecule delimiter character (e.g.,

In some embodiments, the input representation can be encoded for use with the model. For example, the character-set that makes up the input strings can be converted into tokenized strings, such as by replacing letters with integer token representatives (e.g., where each character is replaced with an integer, sequences of characters are replaced with an integer, and/or the like). In some embodiments, the string of integers can be transformed into one-hot encodings, which can be used to represent a set of categories in a way that essentially makes each category's representation equidistant from other categories. One-hot encodings can be created, for example, by initializing a zero vector of length n, where n is the number of unique tokens in the model's vocabulary. At the position of the token's value, a zero can be changed to a one to indicate the identity of that token. A one-hot encoding can be converted back into a token using a function such as the argmax function (e.g., which returns the index of the largest value in an array). As a result, such encodings can be used to provide a probability distribution over all possible tokens, where 100% of the probability is on the token that is encoded. Accordingly, the output of the model can be a prediction of the probability distribution over all of the possible tokens.

According to some embodiments, the training can require augmenting the training reactions. For example, the input source strings can be augmented for training. As an illustrative example not intended to be limiting, the following example is provided in the context of SMILES notation, although it should be appreciated that any format can be used without departing from the spirit of the techniques described herein. In some embodiments, the augmentation techniques can include performing non-canonicalization. SMILES represents molecules as a traversal of the molecular graph. Most graphs have more than one valid traversal order, which can be analogized to the idea of a “pose” or view from a different direction. SMILES can have canonical traversal orders, which can allow for a single, unique representation for each molecule. Since a number of noncanonical SMILES can represent the same molecule, the techniques can produce a variety of different input strings that represent the same information. In some embodiments, a random noncanonical SMILES is produced for each molecule each time it is used during training. Since each molecule can be used a number of different times during training, the techniques can generate a number of different noncanonical SMILES for each molecule, which can make the model robust and able to handle variations in the input.

In some embodiments, the augmentation techniques can include performing a chirality inversion. Chemical reactions can be mirror symmetric, such that mirroring the molecules of a reaction can result in another valid reaction example. Such mirroring techniques can produce new training examples if there is at least one chiral center in the reaction, and therefore mirrored reactions can be generated for inputs with at least one chiral center. As a result, for any reaction containing a chiral center, the reaction can be inverted to create a mirrored reaction before training (e.g., by inverting all chiral centers of the reaction). Such techniques can mitigate bias in the training data where classes of reactions may have predominantly more examples with one chirality than another.

In some embodiments, the augmentation techniques can include performing an agent dropout. Frequently, examples in the dataset are missing agents (e.g., solvents, catalysts, and/or reagents). During training, agent molecules can be omitted in the reaction example, which can make the model more robust to missing information during inference. In some embodiments, the augmentation techniques can include performing molecule order shuffling. For example, the order that input molecules are listed can be irrelevant to the prediction. As a result, the techniques can include randomizing the order of the input molecules (e.g., for each input during training).

While the entire data set can be augmented prior to training, the inventors have appreciated that such an approach can result in a much longer training time since all of the data must first be augmented, and then the training occurs afterwards, such that the training cannot be done in parallel with any of the augmentation. Therefore, the inventors have developed techniques of incrementally augmenting the set of reactions used for training that can be used in some embodiments. In particular, the techniques can include augmenting a subset of the training data, and then using that augmented subset to start training the models while other subset(s) of the training data are augmented for training. For example, for a forward prediction model, the model can be trained using the augmented subset of training reactions by using the products of the augmented reactions as inputs and the sets of reactions of the augmented reactions as the output. The training process can continue as each subset of training data is augmented accordingly. As another example, for a reverse prediction model, the model can be trained using the sets of reactions of the augmented reactions as input and the products of the reactions as output, which can be performed iteratively for each augmented subset.

Reaction conditions can be useful information for implementing a suggested synthetic route. However, chemists typically are left to turn to literature to find a methodology used in similar reactions to help them design the procedure they will attempt themselves. This can be suboptimal, for example, because chemists must spend time surveying literature, make subjective decisions about which reactions are similar enough to be relevant, and in cases involving automation, convert the procedure into a detailed algorithm for machines to carry out, etc.

The techniques described herein can include providing, e.g., by extending concepts of a molecular transformer, a list of actions in a machine-readable format. Referring further to FIG. 2, in some embodiments the prediction engine 202 can generate an action prediction 212. For example, a reverse model can predict the reactants/agents as described herein, followed by a list of actions. In some embodiments, the list of actions can be provided in a structured text format, such as JSON/XML/HTML. It should be appreciated that use of a structured text format can run against conventional wisdom, as structured data is often considered to lead to inferior models (e.g., compared to natural language approaches). However, the inventors have appreciated that structured text formats can be used in conjunction with the techniques described herein without such conventional problems. The forward model can read in the reactants/agents predicted by the reverse model with the action list, and use it to predict the product molecule. The action list may repeat the SMILES strings of molecules already being specified in the reactants/agents. Conceptually, this is similar to the idea of a materials and methods section of an academic paper, where the required materials listed first, followed by the procedure which utilizes them. Due to imperfections in the data, not all molecules/species in the reactants/agents may be found in the action list (and vice versa). Therefore, in some embodiments, the techniques can include the reactant/agents and action list together. If such imperfections in the data are not present, then in some embodiments the reactants/agents could be omitted for the sake of brevity.

In some embodiments, the techniques can include training a model to predict the natural language procedure associated with a given reaction. Referring again to FIG. 2, in some embodiments the prediction engine 202 can generate a procedure 214 accordingly. This can be useful, in some scenarios, since such techniques need not rely on an algorithm (e.g., which may cause errors) to convert a reaction paragraph into a structured action list. Aspects of chemical procedures can be difficult to express in a simplified list format. Therefore, in some embodiments, the techniques can include replacing molecule/species names with their SMILES equivalent, which can allow the model to simply transcribe the relevant molecules where appropriate when writing the procedure. Without this change, for example, the model would need to learn to translate

SMILES into all varieties of different chemical nomenclature present in the data (e.g., IUPAC, common names, reference indices), which could limit its generalizability. Additionally, small details that may be discarded when converting to an action list can instead be retained (e.g., the product was obtained as a colorless oil). The generation of a natural language procedure can provide for easier interactions for chemists to interact with the techniques described herein, since it can be done through a format that chemists are used to reading (e.g., procedures in literature/patents).

### Example Algorithm Flow

Without intending to limit the techniques described herein, below is an example training and prediction process for constructing a chemical reaction network using the techniques described herein.

### Training

The training input includes a set of training reactions (e.g., in a database or list of chemical reactions). The set of training reactions can include, for example, millions of reactions taken from US patents, such as approximately three million reactions. The reactions can be read in any format or notation, as described herein. A single-step retrosynthesis model can be trained using the molecular transformer model, such as similar to that described in Segler, which is incorporated herein, with the products in the training dataset as input and the corresponding reactants as output. Modifications to the model described in Segler can include, for example, using a different optimizer (e.g., Adamax), a different learning rate (e.g., 5.e⁻⁴ for this example), a different learning rate warmup schedule (e.g., linear warm up from 0 to 5.e⁻⁴ over 8,000 training iterations), no learning rate decay, and a longer training duration (e.g., five to ten times that described in Segler), and/or the like.

### Execution

The input to execute the prediction engine is a target molecule fingerprint (e.g., again as SMILES, SMARTS, and/or any other fingerprint notations). The ultimate output is the chemical reaction network or graph, which can be generated using the following exemplary steps:

Step 1—receive and/or read in input target molecule fingerprint.

Step 2—execute a graph traversal thread to make periodic requests for single-step retrosynthesis target molecules.

Step 3—execute molecule expansion (single-step prediction) thread(s) to fulfill prediction requests from the graph traversal thread. As described herein multiple molecule expansion threads can be executed, since the runtime performance can scale (e.g., linearly) with the number of single-step prediction threads.

Step 4—collect all unique reactions predicted by molecule expansion thread(s).

Step 5—for each reactant set in the reactions collected from Step 4, collect the new reaction outputs by recursively repeating Steps 2-4 until reaching one or more predetermined criteria, such as performing a specified number of molecule expansions and/or reaching any other relevant criteria reached such as time limit, identifying desired starting materials, identifying desired reactions, and/or the like.

Step 6—the list of reactions collected from iteratively performing steps 2-5 contains all the information needed to determine the chemical reaction network or graph.

Step 7—return chemical reaction network or graph.

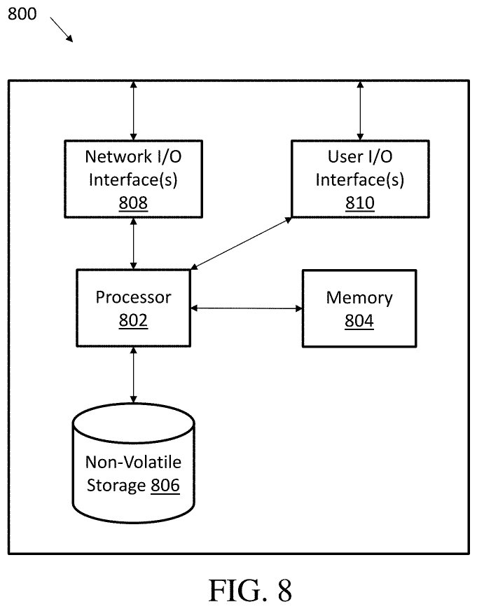

The techniques described herein can be incorporated into various types of circuits and/or computing devices. FIG. 8 shows a block diagram of an exemplary computer system 800 that may be used to implement embodiments of the technology described herein. For example, the computer system 800 can be an example of the user computing device 102 and/or the remote computing device(s) 104 in FIG. 1. The computing device 800 may include one or more computer hardware processors 802 and non-transitory computer-readable storage media (e.g., memory 804 and one or more non-volatile storage devices 806). The processor(s) 802 may control writing data to and reading data from (1) the memory 804; and (2) the non-volatile storage device(s) 806. To perform any of the functionality described herein, the processor(s) 802 may execute one or more processor-executable instructions stored in one or more non-transitory computer-readable storage media (e.g., the memory 804), which may serve as non-transitory computer-readable storage media storing processor-executable instructions for execution by the processor(s) 802. The computing device 800 also includes network I/O interface(s) 808 and user I/O interfaces 810.

U.S. Provisional Application Ser. No. 63/140,090, filed on Jan. 21, 2021, entitled “SYSTEMS AND METHODS FOR TEMPLATE-FREE REACTION PREDICTIONS,” is incorporated herein by reference in its entirety.

The terms “program” or “software” are used herein in a generic sense to refer to any type of computer code or set of processor-executable instructions that can be employed to program a computer or other processor (physical or virtual) to implement various aspects of embodiments as discussed above. Additionally, according to one aspect, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform tasks or implement abstract data types. Typically, the functionality of the program modules may be combined or distributed.

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, for example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements);etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term). The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

What is claimed is:

1. A computerized method for determining a set of reactions to produce a target product, the method comprising:
receiving the target product;
executing a graph traversal thread;
requesting, via the graph traversal thread, a first set of reactant predictions for the target product;
executing a molecule expansion thread;
determining, via the molecule expansion thread and a reactant prediction model, the first set of reactant predictions; and
storing the first set of reactant predictions as at least part of the set of reactions.

2. The method of claim 1, further comprising:
requesting, via the graph traversal thread, a second set of reactant predictions for a reactant prediction from the first set of reactant predictions;
executing a second molecule expansion thread; and
determining, via the second molecule expansion thread and the reactant prediction model, the second set of reactant predictions.

3. The method of claim 2, further comprising storing the second set of reactant predictions with the first set of reactant predictions as at least part of the set of reactions.

4. The method of claim 1, further comprising:
accessing a set of training reactions; and
training the reactant prediction model using the set of training reactions.

5. The method of claim 4, wherein training the reactant prediction model using the set of training reactions comprises incrementally augmenting the set of training reactions during training.

6. The method of claim 5, wherein incrementally augmenting the set of training reactions comprises:
augmenting a first portion of the set of training reactions; and
training the reactant prediction model using the augmented first portion of the set of training reactions, comprising using, for each training reaction in the augmented first portion:
a product of the training reaction as an input; and
a set of reactions of the training reaction as an output.

7. The method of claim 6, wherein incrementally augmenting the set of training reactions comprises:
augmenting a second portion of the set of training reactions; and
training the reactant prediction model using the augmented second portion of the set of training reactions, comprising using, for each training reaction in the augmented second portion:
a product of the training reaction as the input; and
a set of reactions of the training reaction as the output.

8. The method of claim 5, wherein incrementally augmenting the set of training reactions comprises:
augmenting a first portion of the set of training reactions; and
training the reactant prediction model using the augmented first portion of the set of training reactions, comprising using, for each training reaction in the augmented first portion:
a set of reactions of the training reaction as an input; and
a product of the training reaction as an output.

9. The method of claim 8, wherein incrementally augmenting the set of training reactions comprises:
augmenting a second portion of the set of training reactions; and
training the reactant prediction model using the augmented second portion of the set of training reactions, comprising using, for each training reaction in the augmented second portion:
a set of reactions of the training reaction as the input; and
a product of the training reaction as the output.

10. The method of claim 1, further comprising executing an orchestrator thread, wherein the orchestrator thread:
executes the graph traversal thread;
receives, via the graph traversal thread, the request for the first set of reactant predictions for the target product; and
executes the molecule expansion thread to determine the first set of reactant predictions.

11. The method of claim 10, wherein the orchestrator thread transmits the determined first set of reactant predictions to the graph traversal thread.

12. The method of claim 10, wherein the orchestrator thread stores the first set of reactant predictions to maintain a retrosynthesis graph.

13. The method of claim 12, further comprising executing a tree search on the retrosynthesis graph to identify a set of possible routes through the retrosynthesis graph, wherein each route of the set of possible routes represents an associated way to build the target product.

14. The method of claim 13, further comprising updating, for each route identified in the set of possible routes, a blacklist of reactant-product pairs.

15. The method of claim 14, further comprising omitting one or more additional routes from the set of possible routes by determining, during the tree search, that the one or more additional routes containing a reaction in a reaction-product pair in the blacklist.

16. The method of claim 1, wherein the reactant prediction model is a trained single-step retrosynthesis model that determines the first set of reactant predictions based on the target product.

17. The method of claim 16, wherein the single-step retrosynthesis model comprises:
a trained forward prediction model configured to generate a product prediction based on a set of input reactants; and
a trained reverse prediction model configured to generate a set of reactant predictions based on an input product.

18. The method of claim 17, wherein the set of input reactants, the set of reactant predictions, or both, comprise one or more of:
one or more reagents;
one or more catalysts; and
one or more solvents.

19. The method of claim 17, wherein determining, via the reactant prediction model, the first set of reactant predictions comprises:
predicting, by running the trained reverse prediction model on the target product, the first set of reactant predictions;
predicting, by running the trained forward prediction model on the first set of reactant predictions, a product; and
comparing the target product with the predicted product to determine whether to store the first set of reactant predictions.

20. A non-transitory computer-readable media comprising instructions that, when executed by one or more processors on a computing device, are operable to cause the one or more processors to determine a set of reactions to produce a target product by performing:
receiving the target product;
executing a graph traversal thread;
requesting, via the graph traversal thread, a first set of reactant predictions for the target product;
executing a molecule expansion thread;
determining, via the molecule expansion thread and a reactant prediction model, the first set of reactant predictions; and
storing the first set of reactant predictions as at least part of the set of reactions.

21. A system comprising a memory storing instructions, and at least one processor configured to execute the instructions to determine a set of reactions to produce a target product by performing:
receiving the target product;
executing a graph traversal thread;
requesting, via the graph traversal thread, a first set of reactant predictions for the target product;
executing a molecule expansion thread;
determining, via the molecule expansion thread and a reactant prediction model, the first set of reactant predictions; and
storing the first set of reactant predictions as at least part of the set of reactions.
